(19) United States
(12) Patent Application Publication
(10) Pub. No.: US 2021/0145423 A1
(43) Pub. Date: May 20, 2021

(54) Implantation Systems and Related Methods of Implanting Tissue Stimulators

(71) Applicant: Stimwave Technologies Incorporated, Pompano Beach, FL (US)

(72) Inventors: Laura Tyler Perryman, Pompano Beach, FL (US); Chad David Andresen, Miami Beach, FL (US)

(21) Appl. No.: 17/099,530

(22) Filed: Nov. 16, 2020

Related U.S. Application Data
(60) Provisional application No. 62/935,398, filed on Nov. 14, 2019.

(51) Int. Cl. A61B 17/00; A61N 1/05; A61N 1/375; A61B 17/29

(52) U.S. Cl. CPC A61B 17/00234; A61N 1/0558; A61B 2017/00336; A61B 17/29; A61B 2017/2904; A61N 1/37518

(57) ABSTRACT

A method of percutaneously implanting a tissue stimulator at a tissue of a body includes inserting a sheath of an implantation device into the body through an incision adjacent the tissue and inserting the tissue stimulator into the sheath to position one or more fixation mechanisms of the tissue stimulator near the tissue within the body. With at least a portion of the sheath inside of the body, the method further includes separating the sheath into two or more portions and moving the two or more portions apart from each other to remove the sheath from the tissue stimulator. The method further includes withdrawing the two or more portions of the sheath from the body through the incision.

### TECHNICAL FIELD

This disclosure relates to implantation systems and related methods of implanting tissue stimulators, such as methods of percutaneously implanting neurostimulators using splittable introducers.

### BACKGROUND

Modulation of tissue within the body by electrical stimulation has become an important type of therapy for treating chronic, disabling conditions, such as chronic pain, problems of movement initiation and control, involuntary movements, dystonia, urinary and fecal incontinence, sexual difficulties, vascular insufficiency, and heart arrhythmia. For example, an external antenna can be used to send electrical energy to electrodes on an implanted tissue stimulator that can pass pulsatile electrical currents of controllable frequency, pulse width, and amplitudes to a tissue.

### SUMMARY

In general, this disclosure relates to implantation systems and related methods of percutaneously implanting neurostimulators using splittable introducers, and to self-fixating neurostimulators and fixation deployment assistance tools.

In one aspect, an implantation system includes a tissue stimulator and an implantation device. The tissue stimulator includes a fixation module configured to anchor the tissue stimulator to a tissue. The implantation device includes a sheath that defines a passage for receiving the tissue stimulator to position the tissue stimulator at the tissue, a curved interior surface at an opening of the passage for forcing the fixation module of the tissue stimulator into a collapsed configuration in which the tissue stimulator is movable through the passage, and a handle by which the sheath is breakable into two or more portions for removing the sheath from the tissue stimulator to allow the fixation module to adjust to an extended configuration in which the fixation module can grip the tissue to anchor the tissue stimulator to the tissue.

In another aspect, a method of percutaneously implanting a tissue stimulator at a tissue of a body includes inserting a sheath of an implantation device into the body through an incision adjacent the tissue. The method further includes inserting the tissue stimulator into the sheath to position one or more fixation mechanisms of the tissue stimulator near the tissue within the body. With at least a portion of the sheath inside of the body, the method further includes and separating the sheath into two or more portions and moving the two or more portions apart from each other to remove the sheath from the tissue stimulator. The method further includes withdrawing the two or more portions of the sheath from the body through the incision.

In another aspect, a method of percutaneously implanting a tissue stimulator into a body includes inserting a distal end of the tissue stimulator through an incision near a tissue to be stimulated, positioning electrodes of the tissue stimulator carried at the distal end of the tissue stimulator adjacent the tissue to be stimulated, inserting a sheath of an implantation device into the body through the incision, and inserting a proximal end of the tissue stimulator into the sheath to position fixation mechanisms of the tissue stimulator in an appropriate tissue within the body. With at least a portion of the sheath inside of the body, the method further includes breaking the sheath apart into two or more portions to remove the sheath from the tissue stimulator and withdrawing the two or more portions of the sheath from the body through the incision.

### DESCRIPTION OF DRAWINGS

FIG. 1 is a side view of a tissue stimulator.

FIG. 2A is a side view of an example pointed tip of a protrusion of an anchor body of the tissue stimulator of FIG. 1.

FIG. 2B is a side view of an example rounded tip of a protrusion of an anchor body of the tissue stimulator of FIG. 1.

FIG. 3A is a front view of an example anchor body of the tissue stimulator of FIG. 1 including three protrusions.

FIG. 3B is a front view of an example anchor body of the tissue stimulator of FIG. 1 including four protrusions.

FIG. 4A is a perspective view of an implantation device.

FIG. 4B is a side cutaway view of an outer sheath of the implantation device of FIG. 4A with the tissue stimulator of FIG. 1 in an extended configuration.

FIG. 5 is a cross-sectional view of the outer sheath of FIG. 4B through a lip of the outer sheath.

FIG. 6 is a side cutaway view of the outer sheath of FIG. 4B with the tissue stimulator of FIG. 1 in a collapsed configuration.

FIGS. 7-12 illustrate a method of percutaneously implanting the tissue stimulator of FIG. 1 at a tissue using the implantation device of FIG. 4A at a single incision site.

FIG. 13 illustrates a step involved in percutaneously implanting the tissue stimulator of FIG. 1 at a tissue adjacent an incision site using a gripping tool.

FIG. 14 illustrates a front view of jaws of the gripping tool of FIG. 13.

FIG. 15 illustrates a subsequent step involved in the method illustrated in FIG. 13.

FIG. 16 illustrates a cross-sectional view of an outer sheath surrounding the tissue stimulator of FIG. 1 and the gripping tool of FIG. 13 at the method step illustrated in FIG. 13.

FIGS. 17 and 18 illustrate subsequent steps involved in the method illustrated in FIG. 13.

FIGS. 19-21 illustrate a method of percutaneously implanting the tissue stimulator of FIG. 1 at a tissue adjacent an single incision site using a grasping tool.

FIGS. 22-27 illustrate a method of percutaneously implanting the tissue stimulator of FIG. 1 at a tissue adjacent an incision site using a tunneling sheath.

FIG. 28 is a system block diagram of a neural stimulation system including an embodiment of the tissue stimulator of FIG. 1.

FIG. 29 is a detailed block diagram of the neural stimulation system of FIG. 28.

### DETAILED DESCRIPTION

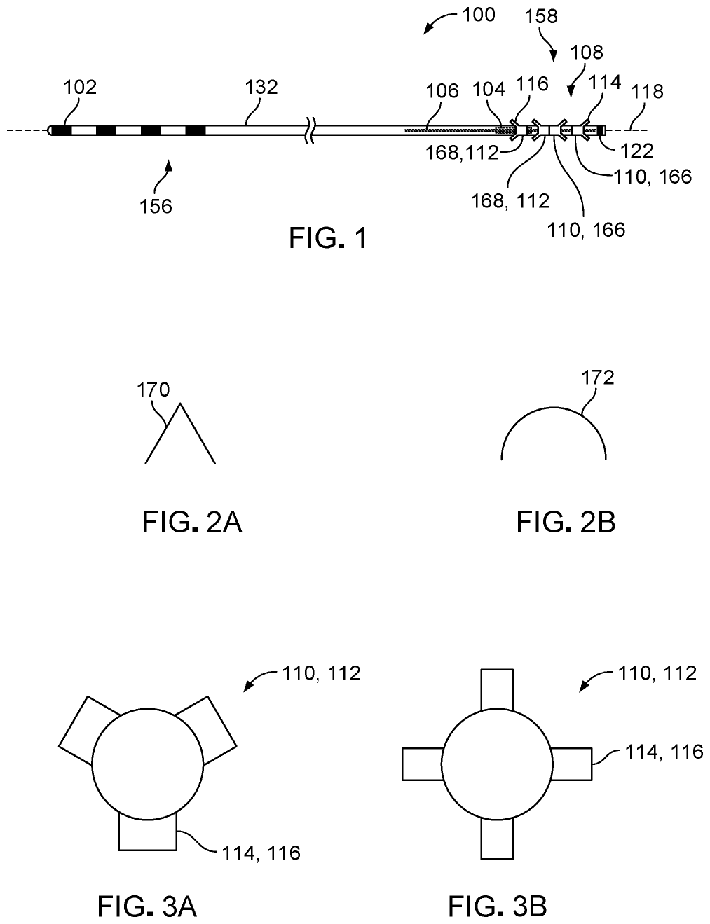

FIG. 1 illustrates a tissue stimulator 100 of designed to be implanted within a patient's body for delivering electrical therapy to tissues within the body. The tissue stimulator 100 includes electrodes 102, an embedded circuit 104 to process wireless energy through an embedded receiver antenna 106, or in some embodiments, an external receiver component. The tissue stimulator 100 further includes a fixation module 108 that is located proximal of the circuit 104 along a body 132 of the tissue stimulator 100. The tissue stimulator 100 also includes a marker band 122.

The fixation module 108 includes one or more anchor bodies 110 and one or more anchor bodies 112. The anchor bodies 110, 112 are tissue fixation mechanisms that respectively include tubular portions 166, 168 and protrusions 114, 116 that extend radially outward from the tubular portions 166, 168. The protrusions 114, 116 may be integrally formed with the tubular portions 166, 168 or formed as separate components that are assembled with the tubular portions 166, 168 during manufacture. The protrusions 114, 116 are oriented at an acute angle with respect to an axis 118 of the tissue stimulator 100. The protrusions 114 are directed in a proximal direction, whereas the protrusions 116 are directed in a distal direction. The oppositely directed protrusions 114, 116 exert counteracting forces on a surrounding tissue to prevent movement of the tissue stimulator 100 within the tissue when the tissue stimulator 100 is implanted within the tissue. Referring to FIG. 2A, in some embodiments, one or more of the protrusions 114, 116 may have a pointed tip 170 that is designed to bite into surrounding adipose tissue. Referring to FIG. 2B, in some embodiments, one or more of the protrusions 114, 116 may have a rounded tip 172 providing more surface area that is better suited to lock into surrounding fascial fibrous tissue. In some embodiments, as shown in FIGS. 3A and 3B, one or more of the protrusions 114, 116 may have tips with a substantially square or otherwise rectangular shape. Other tip shapes are also possible.

Referring still to FIGS. 3A and 3B, the protrusions 114, 116 are typically distributed approximately evenly around a circumference of the anchor bodies 110, 112. In some embodiments, each anchor body 110, 112 includes three protrusions 114, 116, as shown in FIG. 3A. In some embodiments, each anchor body 110, 112 includes four protrusions 114, 116, as shown in FIG. 3B. The anchor bodies 110, 112 of the fixation module 108 are flexible such that the protrusions 114, 116 can be collapsed radially inward towards the body 132 when the tissue stimulator 100 is passed into an introducer sheath to be delivered to an implantation site (as shown in FIG. 6) and such that the protrusions 114, 116 can subsequently rebound back to their initial protrusive orientations (shown in FIG. 1) once the tissue stimulator 100 exits the introducer to engage surrounding tissue at the implantation site.

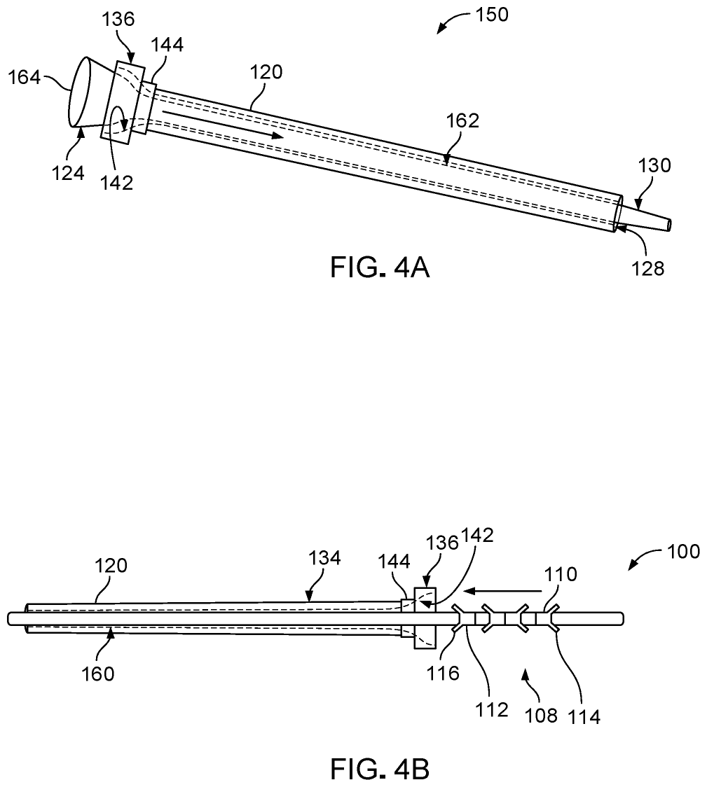

FIG. 4A illustrates an implantation device 150 (e.g., an introducer) that is used to implant the tissue stimulator 100 within the body of a patient. The implantation device 150 and the tissue stimulator 100 together provide an implantation system. The implantation device 150 includes an outer sheath 120 (for example, a tunneler) that is used to deliver the tissue stimulator 100 percutaneously to an implantation site within a patient and that is typically made of plastic. The implantation device 150 also includes a central stylet 124 that provides rigidity and shape to the outer sheath 120 for placement of the outer sheath 120 within the body. The central stylet 124 is typically made of metal.

The central stylet 124 defines an elongate main body 162, a proximal handle 164 that tapers to the main body 162, and a tapered distal tip 130 that extends from the main body 162. The tip 130 may have one or both of blunt features and sharp features. In some examples, a blunt tip facilitates safe insertion of the introducer toward a nerve without damaging (for example, cutting or tearing) the nerve or surrounding vessels, veins, or arteries. In some examples, a sharp tip facilitates subcutaneous delivery of the tissue stimulator 100 at locations that lack large critical arteries or nerves. Additionally, a sharp tip can also facilitate relatively quick delivery of the tissue stimulator 100 to the implantation site and with relatively little effort from an operator when used in subcutaneous spaces.

The metal composition of the central stylet 124 provides rigidity and memory to the outer sheath 120 when the operator bends or otherwise shapes the outer sheath 120 for accessing specific target sites within the body. In contrast, the outer sheath 126 is flexible and does not counteract any bends or memory shapes imposed on the central stylet 124. This provides a significant advantage over conventional introducers that include both a metal stylet and a metal outer sheath, where such a metal central stylet cannot be later extracted from the introducer to leave the metal outer sheath in place.

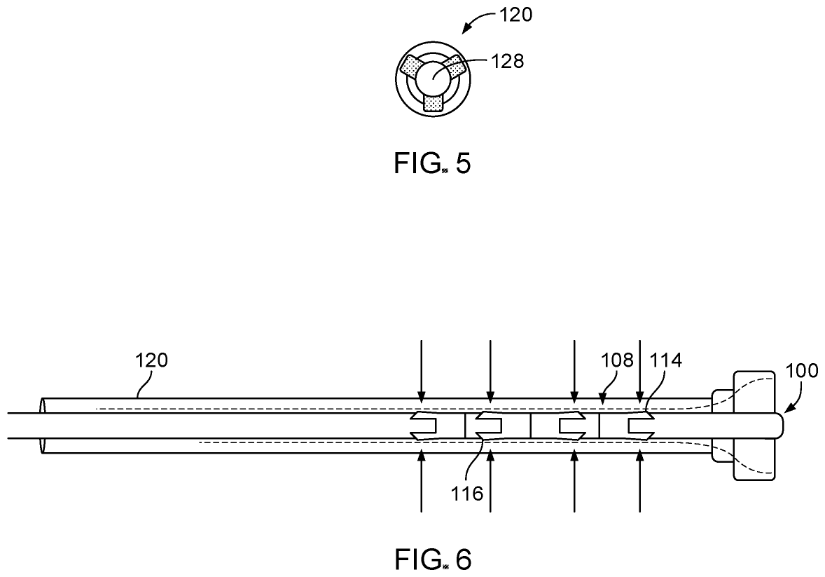

The outer sheath 120 is a generally tubular structure that defines an axial channel 128 that is sized to receive the central stylet 124 and the tissue stimulator 100. The outer sheath 120 further defines a main body 134 and a handle 136 that is integral with the main body 134. The outer sheath 120 also includes a lip 144 that transitions the handle 136 to the main body 134. Referring to FIGS. 4A-6, the handle 136 defines a curved inner profile 142 that causes the protrusions 114, 116 to collapse toward the body 132 of the tissue stimulator 100 upon contact between the protrusions 116 and the outer sheath 120. The curved inner profile 142 is designed to prevent the protrusions 116 from getting caught on an inner surface of the lip 144 and thereby prevents the protrusions 116 from being folded backward upon themselves in an unintended orientation.

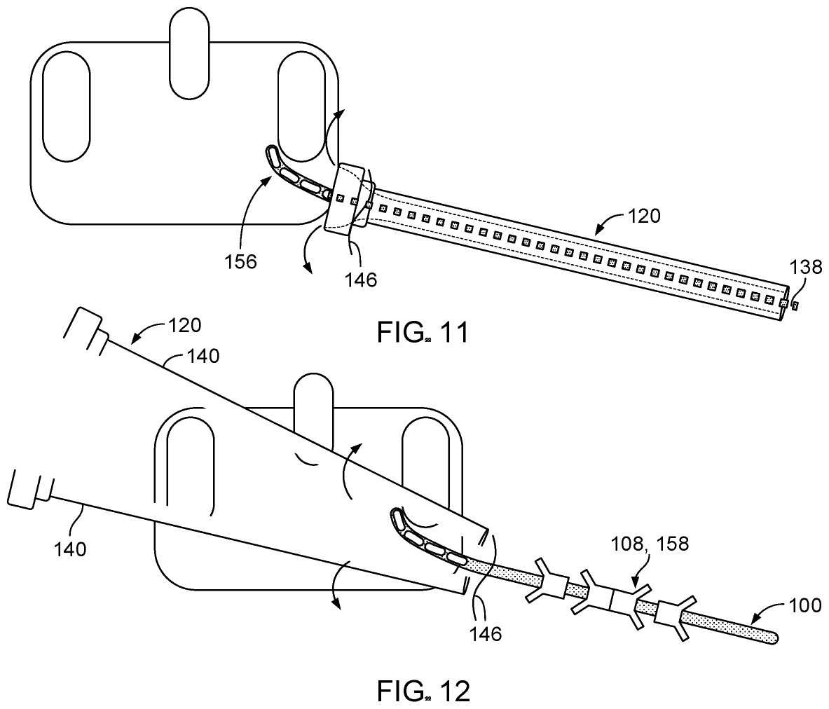

The handle 136 can be manipulated to break (for example, tear) the outer sheath 120 apart into two halves 140 along a linear perforation 138 (shown in FIG. 11). For example, the outer sheath 120 can be ripped at the handle 136 in a way that translates a force down the main body 134 to break the outer sheath 120 apart at the linear perforation 138. This feature of the outer sheath 120 enables the operator to fully implant the tissue stimulator 100 percutaneously into the patient using a single incision. For example, conventional introducers do not break apart, and thus two incisions are required to tunnel tubing, catheters, leads, or stimulators through the introducer from a first incision in the skin to a second incision in the skin and to then safely remove the introducer in a whole, intact form.

In some embodiments, an implantation device that is otherwise similar in construction and function to the implantation device 150 may include an outer sheath that does not have the perforation 138. For such embodiments, the handle of the outer sheath may simply be torn apart to rupture the integrity of polymer material that forms the outer sheath such that the tear is translated along a length of the outer sheath.

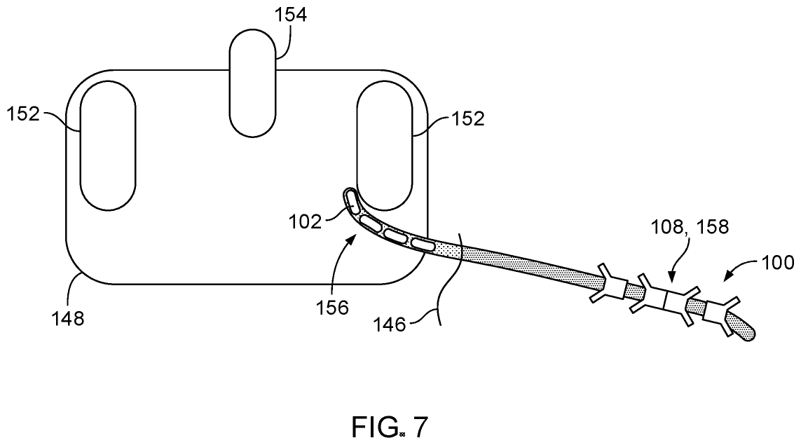

FIGS. 7-12 illustrate an example method of implanting the tissue stimulator 100 percutaneously at a vertebral implantation site using a single incision 146. In the example of FIGS. 7-12, the tissue stimulator 100 is implanted adjacent a vertebral body 148, a pedical 152, and spinous processes 154. In FIG. 7, the incision 146 is made in the skin, and the tissue stimulator 100 is inserted into the incision 146 to position the electrodes 102 in a vicinity of the vertebral body 148. At this stage, a distal portion 156 of the tissue stimulator 100 carrying the electrodes 102 is positioned inside of the body, and a proximal portion 158 of the tissue stimulator 100 carrying the fixation module 108 is positioned outside of the body.

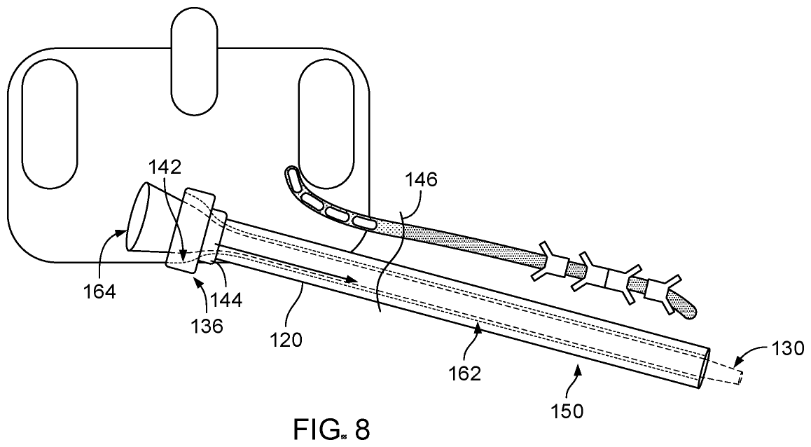

Next, the implantation device 150 is inserted into the incision 146 such that a distal portion 160 of the outer sheath 120 and the distal tip 130 of the central stylet 124 are positioned inside of the body, while the handle 136 of the outer sheath 120 and the handle 164 of the central stylet 124 remain outside of the body, as shown in FIG. 8. Once the outer sheath 120 is positioned at a desired location within the incision 146, the central stylet 124 is grasped by the handle 164 and withdrawn from the outer sheath 120.

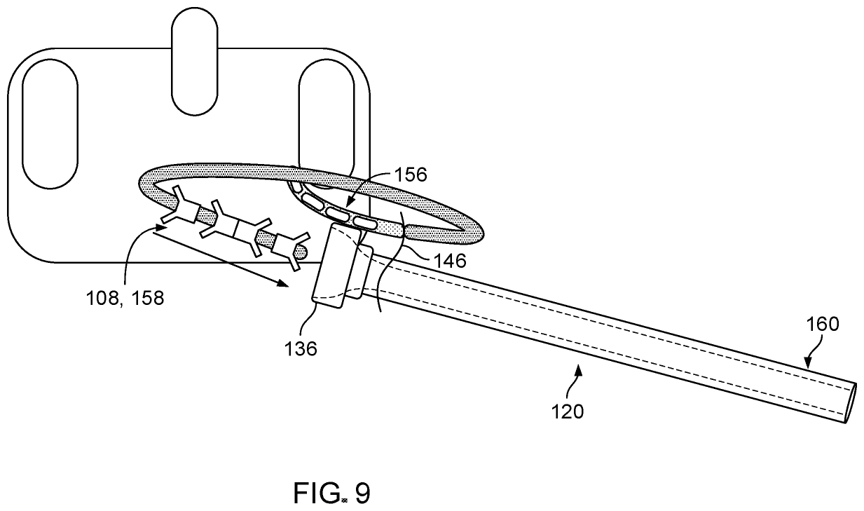

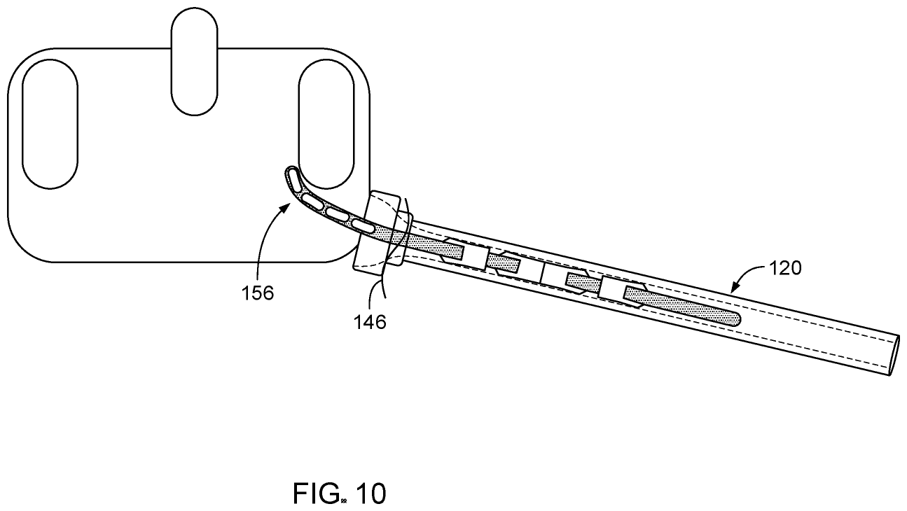

As shown in FIG. 9, the outer sheath 120 is further inserted into the body, and the proximal portion 158 of the tissue stimulator 100 is wrapped around towards the handle 136, which still remains just outside of the body. Referring to FIG. 10, the proximal portion 158 of the tissue stimulator 100, carrying the fixation module 108, is passed into the outer sheath 120, while the distal portion 156 of the tissue stimulator 100 remains within the body near the incision 146 and while the handle 136 of the outer sheath 120 remains outside of the incision 146. The proximal portion 158 of the tissue stimulator 100 is further advanced within the outer sheath 120 until the tissue stimulator 100 is fully extended in a relaxed, linear configuration, as shown in FIG. 10. The electrodes 102 of the tissue stimulator 100 remain positioned near the vertebral body 148. As indicated in FIG. 11, the outer sheath 120 can then be split apart by pulling apart the handle 136.

Referring to FIG. 12, while the tissue stimulator 100 remains inside of the body underneath the incision 146, the outer sheath 120 grasped at the handle 136 and pulled apart into the two halves 140 while also being pulled out of the body through the incision 146. Removal of the outer sheath 120 from the tissue stimulator 100 allows the protrusions 114, 116 to return to their initial, protrusive orientations to grip the surrounding tissue to anchor the tissue stimulator 100 in a fixed position.

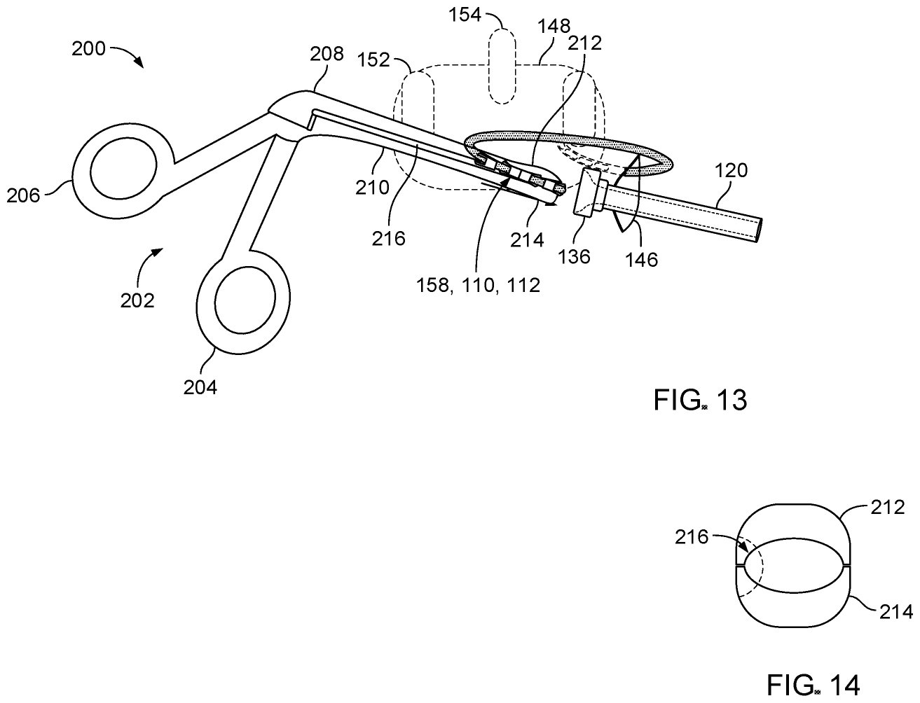

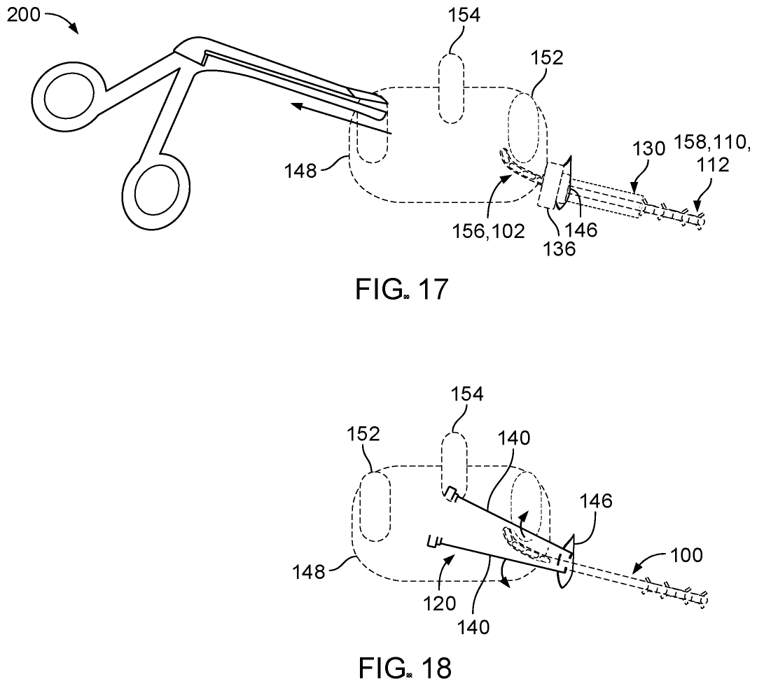

In some implementations, at the stage when both the proximal portion 158 of the tissue stimulator 100 and the handle 136 of the outer sheath 120 are positioned outside of the incision 146, as shown in FIG. 8, the proximal portion 158 of the tissue stimulator 100 may be inserted into the outer sheath 120 using a gripping tool 200, as shown in FIG. 13. The gripping tool 200 includes a handle 202 with two scissor handle portions 204, 206 that extend respectively from two arms 208, 210 of the gripping tool 200. Referring to FIGS. 13 and 14, a pivotable jaw 212 is connected to a distal end of the arm 208, and a distal end of the arm 210 defines a stationary jaw 214. In some embodiments, the handle portion 206 can be moved toward the handle portion 204 to pivot the jaw 212 toward the jaw 214 to grasp the tissue stimulator 100 within the jaws 212, 214, as shown in FIG. 13. In some embodiments, the handle portion 206 can be moved away from the handle portion 204 to pivot the jaw 212 away from the jaw 214 to release the tissue stimulator 100 from the grip of the jaws 212, 214, as shown in FIG. 17.

Referring still to FIGS. 13 and 14, the arms 208, 210 together form a channel 216 that is sized to accommodate the body 132 and the distal portion 156 of the tissue stimulator 100 along at least one side of the tissue stimulator 100 while the proximal portion 158 of the tissue stimulator 100 is gripped within the jaws 212, 214. Gripping the proximal portion 158 of the tissue stimulator 100 within the jaws 212, 214 causes the protrusions 114, 116 of the anchor bodies 110, 112 to collapse towards the body 132 of the tissue stimulator 100 to facilitate placement of the tissue stimulator 100 into the outer sheath 120.

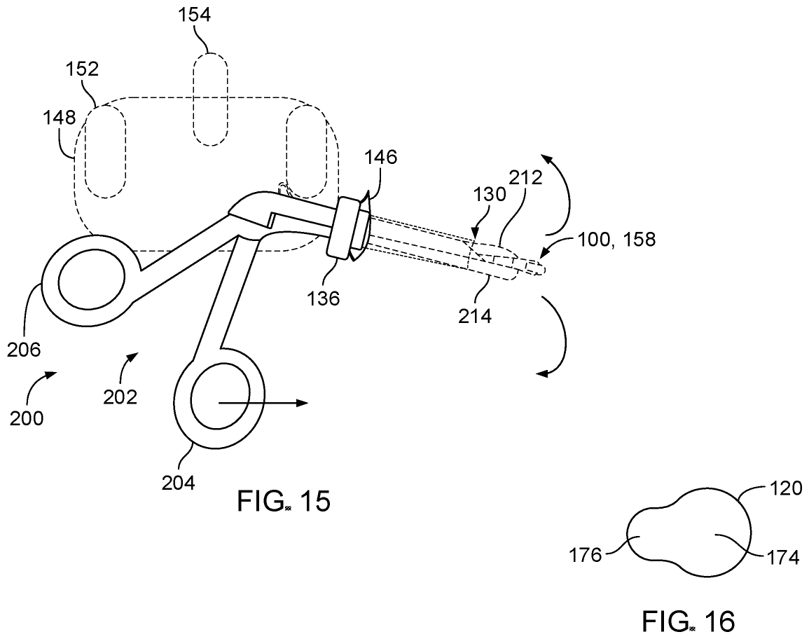

Referring to FIG. 15, the gripping tool 200, with the tissue stimulator 100 gripped within the jaws 212, 214, can be moved further into the outer sheath 120 until the proximal portion 158 of the tissue stimulator 100 extends past the distal tip 130 of the outer sheath 120. Upon movement of the proximal portion 158 past the distal tip 130, the protrusions 114, 116 of the anchor bodies 110, 112 return to their initial, radially outward orientations to secure the tissue stimulator 100 to the surrounding tissue in a fixed position. With both the arms 208, 210 of the gripping tool 200 and the tissue stimulator 100 within the outer sheath 120, the jaws 212, 214 may be opened to release the tissue stimulator 100 as described above, and the gripping tool 200 may be moved laterally with respect to the tissue stimulator 100 for removal of the gripping tool 200 from the outer sheath 120 while the tissue stimulator 100 remains in position within the outer sheath. For example, as shown in FIG. 16, the gripping tool 200 may be moved slightly laterally to a position 174 within the outer sheath, while the tissue stimulator 100 is located at a position 176 within the outer sheath 120.

Referring to FIG. 17, the gripping tool 200 may then be withdrawn from the outer sheath 120, and the outer sheath 120 may be pulled apart and withdrawn through the incision 146, as shown in FIG. 18. In some implementations, the outer sheath 120 may be removed from the body before the gripping tool 200 is removed from the body, or the outer sheath 120 and the gripping tool 200 may be removed from the body concurrently.

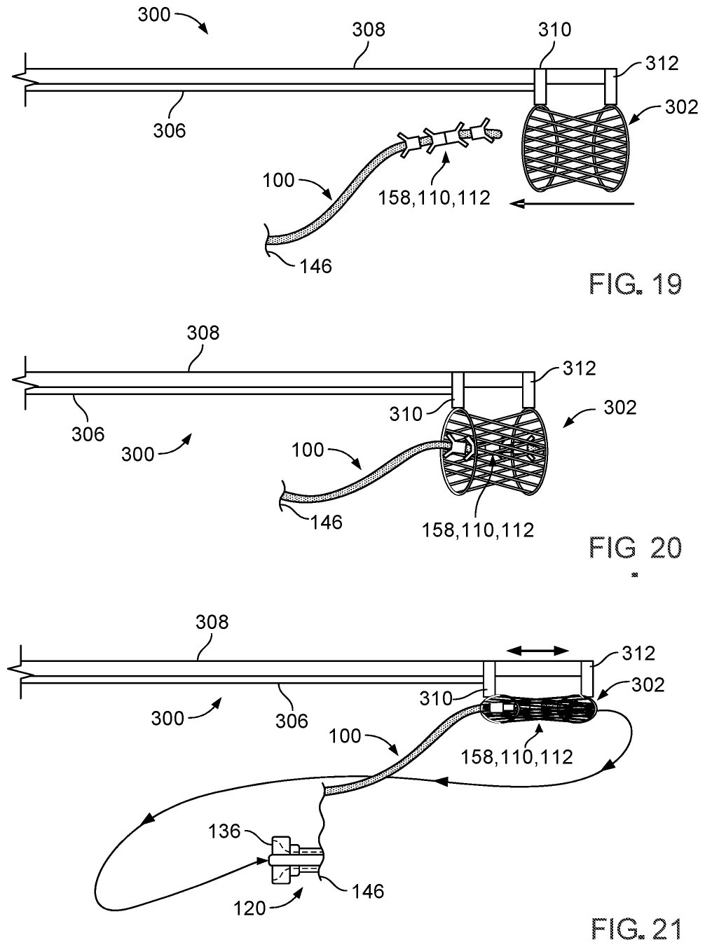

In some implementations, a different type of tool may be used to insert the proximal portion 158 of the tissue stimulator 100 into the outer sheath 120. For example, FIGS. 19-21 illustrate a grasping tool 300 that includes an adjustable net 302 for grasping the proximal portion 158 of the tissue stimulator 100. The grasping tool 300 includes a handle (not shown) from which a lower shaft member 306 and an upper shaft member 308 extend. The shaft members 306, 308 are movable (e.g., slidable) with respect to each other. The net 302 is attached to the lower and upper shafts 306, 308 respectively at connectors 310, 312.

Referring to FIGS. 19 and 20, with the proximal portion 158 of the tissue stimulator 100 and the handle 136 of the outer sheath 120 located outside of the incision 146, the net 302 is moved over the proximal portion 158 until all of the anchor bodies 110, 112 are surrounded by the net 302. Referring to FIG. 21, the lower and upper shaft members 306, 308 are then moved away from each other to cause the net 302 to expand (e.g., lengthen) axially and to reduce in width (e.g., outer diameter) to cinch around the anchor bodies 110, 112, thereby causing the protrusions 114, 116 of the anchor bodies 110, 112 to collapse towards the body 132 of the tissue stimulator 100. With the proximal portion 158 of the tissue stimulator 100 secured within the net 302 and with the protrusions 112, 114 in the collapsed configuration, the grasping tool 300 is moved into the outer sheath 120 until the anchor bodies 110, 112 pass the distal tip 130 of the outer sheath 120 and expand to secure the tissue stimulator 100 to the surrounding tissue. At this stage, the lower and upper shaft members 306, 308 can be moved toward each other to loosen the net 302 around the proximal portion 158 of the tissue stimulator 100, and the gripping tool 300 can then be withdrawn from the body through the incision 146 to leave the tissue stimulator 100 implanted in place. As discussed above, the outer sheath 120 may then be pulled apart and withdrawn from the body through the incision 146.

In alternative embodiments, a grasping tool that is otherwise substantially similar in construction and function to the grasping tool 300 may include a net that is made of one or more dissolvable materials instead of the net 302. Accordingly, the net can dissolve within the body over time and does not need to be removed from the body. Such a grasping tool also includes the shaft members 306, 308, the connectors 310, 312, and a proximal mechanism that is coupled to the connectors 310, 312 for causing the connectors 310, 312 to release the net. For this embodiment, once the proximal portion 158 of the tissue stimulator 100 has been moved past the distal tip 130 of the outer sheath 120, the proximal mechanism is adjusted to cause the connectors 310, 312 to release the net, and the shaft members 306, 308 are withdrawn from the body through the incision 146. As discussed above, the outer sheath 120 may then be pulled apart and withdrawn from the body through the incision 146. Example materials from which the net may be made include resorbable materials (e.g., resorbable suture materials or other types of resorbable materials) and bio-ingrowth promotion materials that may speed up anchoring through scar tissue. Example resorbable materials from which the net may be made include polyglactin 910, polyglycolic acid, glycolide, trimethylene carbonate, poliglecaprone 25, and polylactic acid (PLA). Example bio-ingrowth promotion materials from which the net may be made include bone morphogenic protein (BMP), polyethylene terephthalate (PET), poly(methyl methacrylate) (PMMA), polydimethylsiloxane (PDMS), parylene, polyurethane, ethylene tetrafluororthylene (ETFE), polytetrafluoroethylene (PTFE), and polycarbonate.

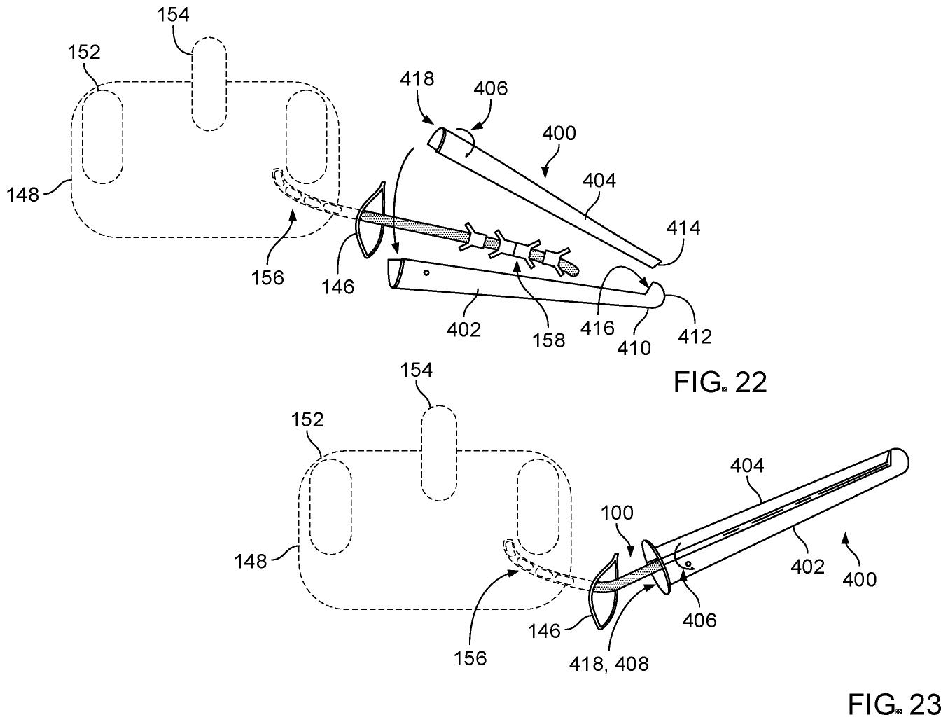

While the above-discussed methods of implanting the tissue stimulator 100 within the body have been described and illustrated with respect to the outer sheath 120 that is designed to be torn apart for removal from the body, in some embodiments, a different type of introducer may be used to implant the tissue stimulator 100. For example, FIGS. 22 and 23 illustrates a novel tunneling sheath 400 that can be used to implant the tissue stimulator 100 within the body. The tunneling sheath 400 is a multi-part (e.g., two-part) device that includes a first support member 402 and a cooperating second support member 404 that can be connected to each other at one end at a latch mechanism 406.

The support members 402, 404 together form a channel 408 that is sized to securely surround at least the proximal portion 158 of the tissue stimulator 100 with the protrusions 114, 116 of the anchor bodies 110, 112 in the collapsed configuration. The first support member 402 defines a tip 410 with an end profile 412 that is optimized (e.g., blunt and rounded) for tunneling through subcutaneous tissue. The second support member 404 defines a flat end 414 that is formed to seat against a flat section 416 of the tip 410. The support members 402, 404 also define rims that together form a handle 418 of the tunneling sheath 400.

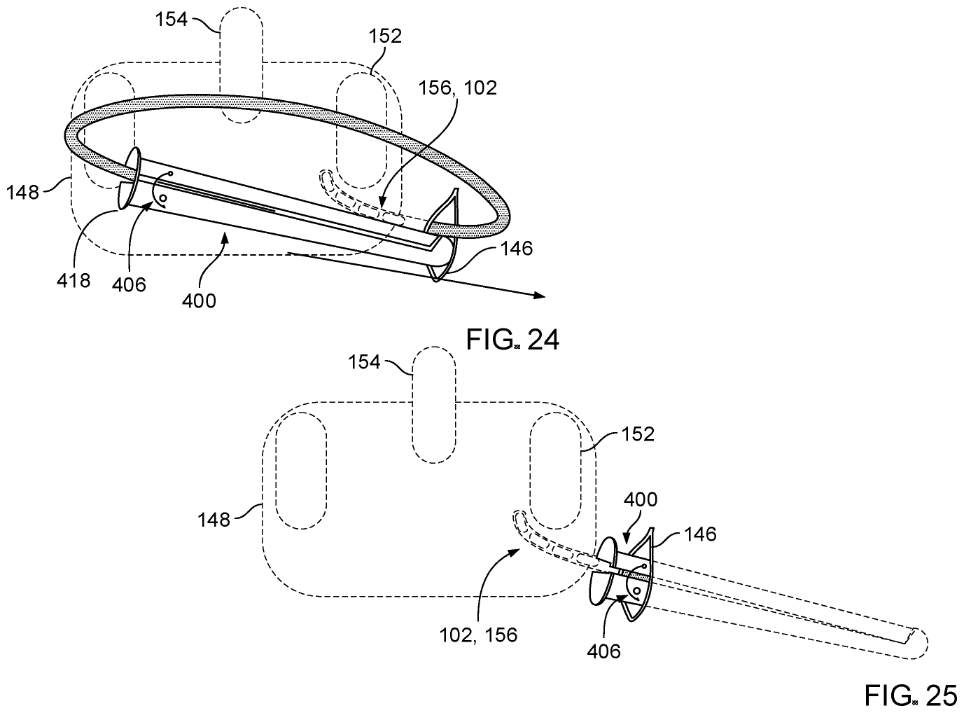

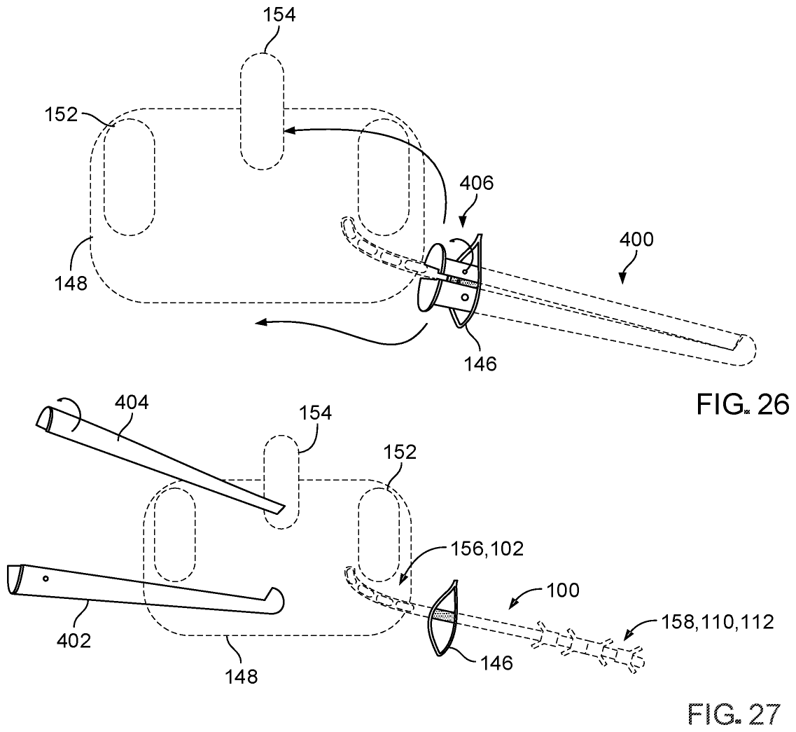

Referring to FIGS. 22 and 23, after the electrodes 102 of the tissue stimulator 100 are positioned adjacent the target tissue to be stimulated, the support members 402, 404 of the tunneling sheath 400 are attached to each other and securely placed around the proximal portion 158 of the tissue stimulator 100. Referring to FIGS. 24 and 25, the tunneling sheath 400, carrying the proximal potion 158 of the tissue stimulator 100, is inserted into the incision 146 and moved to a desired position for the proximal portion 158. Referring to FIG. 26, a latch mechanism 406 of the tunneling sheath 400 may be unhooked to allow splitting apart of the support members 402, 404. Referring to FIG. 27, the support members 402, 404 may be pulled apart and withdrawn through the incision 146 to allow the protrusions 114, 116 of the anchor bodies 110, 112 to rebound to their original shape and accordingly secure the tissue stimulator 100 to the surrounding tissue.

While the tunneling sheath 400 has been described and illustrated as including two support members 402, 404, in some embodiments, a tunneling sheath 400 that is otherwise substantially similar in structure and function to the tunneling sheath 400 may alternatively include more than two support members.

While the above-discussed tissue stimulator 100, implantation tools, and implantation techniques have been described and illustrated with respect to positioning the electrodes 102 of the stimulator 100 adjacent a pedical 152 of a vertebral body 148, in some implementations, any of the above-discussed implantation tools and techniques may be used to implant the tissue stimulator 100 at any of a number of different locations within the body.

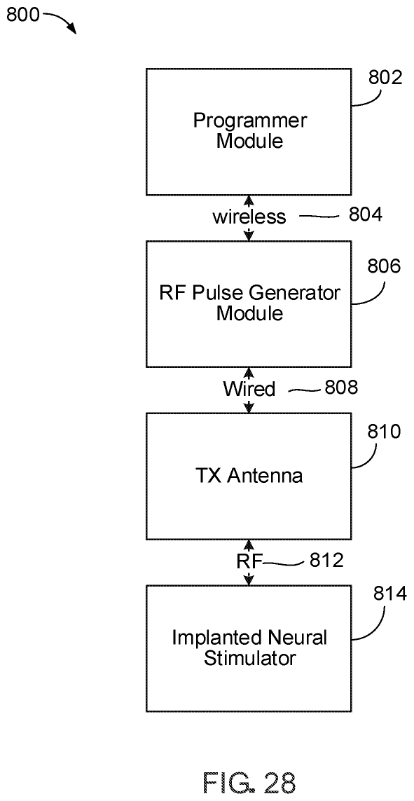

Referring to FIG. 28, the tissue stimulator 100 may be embodied as a tissue stimulator 814 of a neural stimulation system 800. The neural stimulation system further includes a pulse generator 804 that is located exterior to the patient (e.g., handheld by the patient), a transmit (TX) antenna 810 that is connected to the pulse generator 804 and positioned against a skin surface of the patient, and a programmer module 802 that runs a software application. The neural stimulation system 800 is designed to send electrical pulses to a nearby (e.g., adjacent or surrounding) target nerve tissue to stimulate the target nerve tissue by using remote radio frequency (RF) energy without cables and without inductive coupling to power the tissue stimulator 814. Accordingly, the tissue stimulator 814 is provided as a passive tissue stimulator in the neural stimulation system 800. In some examples, the target nerve tissue is in the spinal column and may include one or more of the spinothalamic tracts, the dorsal horn, the dorsal root ganglia, the dorsal roots, the dorsal column fibers, and the peripheral nerves bundles leaving the dorsal column or the brainstem. In some examples, the target nerve tissue may include one or more of cranial nerves, abdominal nerves, thoracic nerves, trigeminal ganglia nerves, nerve bundles of the cerebral cortex, deep brain, sensory nerves, and motor nerves.

In some embodiments, the software application supports a wireless connection 804 (e.g., via Bluetooth®). The software application can enable the user to view a system status and system diagnostics, change various parameters, increase and decrease a desired stimulus amplitude of the electrical pulses, and adjust a feedback sensitivity of the RF pulse generator module 806, among other functions.

The RF pulse generator module 806 includes stimulation circuitry, a battery to power generator electronics, and communication electronics that support the wireless connection 804. In some embodiments, the RF pulse generator module 806 is designed to be worn external to the body, and the TX antenna 810 (e.g., located external to the body) is connected to the RF pulse generator module 806 by a wired connection 808. Accordingly, the RF pulse generator module 806 and the TX antenna 810 may be incorporated into a wearable accessory (e.g., a belt or a harness design) or a clothing article such that electric radiative coupling can occur through the skin and underlying tissue to transfer power and/or control parameters to the tissue stimulator 814.

The TX antenna 810 can be coupled directly to tissues within the body to create an electric field that powers the implanted tissue stimulator 814. The TX antenna 810 communicates with the tissue stimulator 814 through an RF interface. For instance, the TX antenna 810 radiates an RF transmission signal that is modulated and encoded by the RF pulse generator module 806. The tissue stimulator 814 includes one or more antennas (e.g., dipole antennas) that can receive and transmit through an RF interface 812. In particular, the coupling mechanism between the TX antenna 810 and the one or more antennas on the tissue stimulator 814 is electrical radiative coupling and not inductive coupling. In other words, the coupling is through an electric field rather than through a magnetic field. Through this electrical radiative coupling, the TX antenna 810 can provide an input signal to the tissue stimulator 814.

In addition to the one or more antennas, the tissue stimulator 814 further includes internal receiver circuit components that can capture the energy carried by the input signal sent from the TX antenna 804 and demodulate the input signal to convert the energy to an electrical waveform. The receiver circuit components can further modify the waveform to create electrical pulses suitable for stimulating the target neural tissue. The tissue stimulator 814 further includes electrodes that can deliver the electrical pulses to the target neural tissue. For example, the circuit components may include wave conditioning circuitry that rectifies the received RF signal (e.g., using a diode rectifier), transforms the RF energy to a low frequency signal suitable for the stimulation of neural tissue, and presents the resulting waveform to an array of the electrodes. In some implementations, the power level of the input signal directly determines an amplitude (e.g., a power, a current, and/or a voltage) of the electrical pulses applied to the target neural tissue by the electrodes. For example, the input signal may include information encoding stimulus waveforms to be applied at the electrodes.

In some implementations, the RF pulse generator module 806 can remotely control stimulus parameters of the electrical pulses applied to the target neural tissue by the electrodes and monitor feedback from the tissue stimulator 814 based on RF signals received from the tissue stimulator 814. For example, a feedback detection algorithm implemented by the RF pulse generator module 806 can monitor data sent wirelessly from the tissue stimulator 814, including information about the energy that the tissue stimulator 814 is receiving from the RF pulse generator 806 and information about the stimulus waveform being delivered to the electrodes. Accordingly, the circuit components internal to the tissue stimulator 814 may also include circuitry for communicating information back to the RF pulse generator module 806 to facilitate the feedback control mechanism. For example, the tissue stimulator 814 may send to the RF pulse generator module 806 a stimulus feedback signal that is indicative of parameters of the electrical pulses, and the RF pulse generator module 806 may employ the stimulus feedback signal to adjust parameters of the signal sent to the tissue stimulator 814.

In order to provide an effective therapy for a given medical condition, the neural stimulation system 800 can be tuned to provide the optimal amount of excitation or inhibition to the nerve fibers by electrical stimulation. A closed loop feedback control method can be used in which the output signals from the tissue stimulator 814 are monitored and used to determine the appropriate level of neural stimulation current for maintaining effective neuronal activation. Alternatively, in some cases, the patient can manually adjust the output signals in an open loop control method.

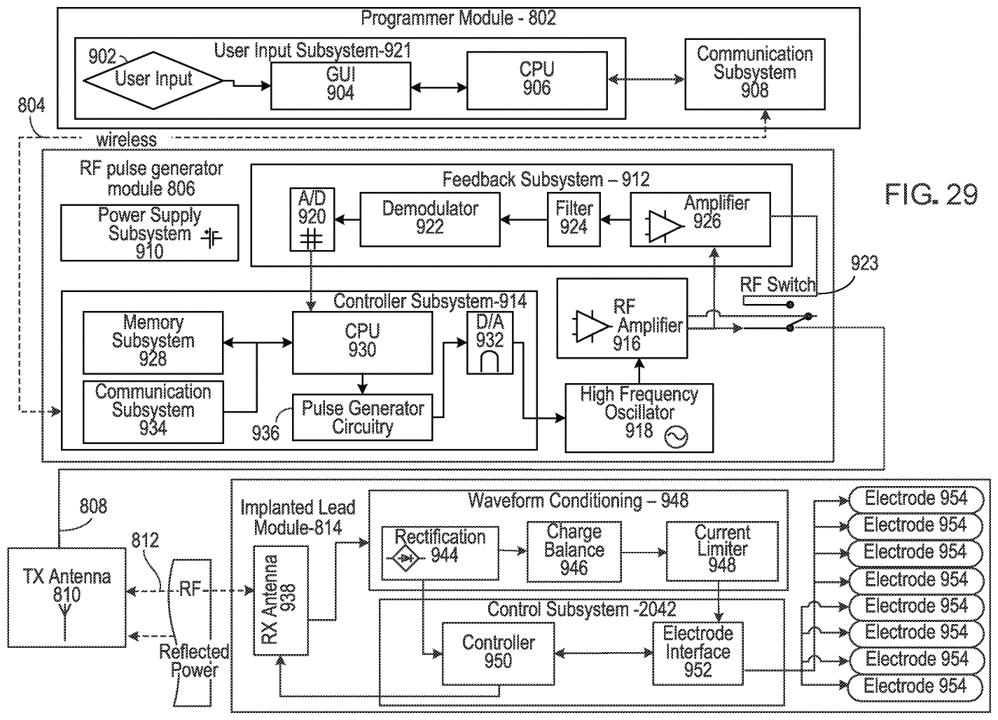

FIG. 29 depicts a detailed diagram of the neural stimulation system 800. The programmer module 802 may be used as a vehicle to handle touchscreen input on a graphical user interface (GUI) 904 and may include a central processing unit (CPU) 906 for processing and storing data. The programmer module 802 includes a user input system 921 and a communication subsystem 908. The user input system 921 can allow a user to input or adjust instruction sets in order to adjust various parameter settings (e.g., in some cases, in an open loop fashion). The communication subsystem 908 can transmit these instruction sets (e.g., and other information) via the wireless connection 804 (e.g., via a Bluetooth or Wi-Fi connection) to the RF pulse generator module 806. The communication subsystem 908 can also receive data from RF pulse generator module 806.

The programmer module 802 can be utilized by multiple types of users (e.g., patients and others), such that the programmer module 802 may serve as a patient's control unit or a clinician's programmer unit. The programmer module 802 can be used to send stimulation parameters to the RF pulse generator module 806. The stimulation parameters that can be controlled may include a pulse amplitude in a range of 0 mA to 20 mA, a pulse frequency in a range of 0 Hz to 2000 Hz, and a pulse width in a range of 0 ms to 2 ms. In this context, the term pulse refers to the phase of the waveform that directly produces stimulation of the tissue. Parameters of a charge-balancing phase (described below) of the waveform can similarly be controlled. The user can also optionally control an overall duration and a pattern of a treatment.

The tissue stimulator 814 or the RF pulse generator module 806 may be initially programmed to meet specific parameter settings for each individual patient during an initial implantation procedure. Because medical conditions or the body itself can change over time, the ability to readjust the parameter settings may be beneficial to ensure ongoing efficacy of the neural modulation therapy.

Signals sent by the RF pulse generator module 806 to the tissue stimulator 814 may include both power and parameter attributes related to the stimulus waveform, amplitude, pulse width, and frequency. The RF pulse generator module 806 can also function as a wireless receiving unit that receives feedback signals from the tissue stimulator 814. To that end, the RF pulse generator module 806 includes microelectronics or other circuitry to handle the generation of the signals transmitted to the tissue stimulator 814, as well as feedback signals received from tissue stimulator 814. For example, the RF pulse generator module 806 includes a controller subsystem 914, a high-frequency oscillator 918, an RF amplifier 916, an RF switch, and a feedback subsystem 912.

The controller subsystem 914 includes a CPU 930 to handle data processing, a memory subsystem 928 (e.g., a local memory), a communication subsystem 934 to communicate with the programmer module 802 (e.g., including receiving stimulation parameters from the programmer module 802), pulse generator circuitry 936, and digital/analog (D/A) converters 932.

The controller subsystem 914 may be used by the user to control the stimulation parameter settings (e.g., by controlling the parameters of the signal sent from RF pulse generator module 806 to tissue stimulator 814). These parameter settings can affect the power, current level, or shape of the electrical pulses that will be applied by the electrodes. The programming of the stimulation parameters can be performed using the programming module 802 as described above to set a repetition rate, pulse width, amplitude, and waveform that will be transmitted by RF energy to a receive (RX) antenna 938 (e.g., or multiple RX antennas 938) within the tissue stimulator 814. The RX antenna 938 may be a dipole antenna or another type of antenna. A clinician user may have the option of locking and/or hiding certain settings within a programmer interface to limit an ability of a patient user to view or adjust certain parameters since adjustment of certain parameters may require detailed medical knowledge of neurophysiology, neuroanatomy, protocols for neural modulation, and safety limits of electrical stimulation.

The controller subsystem 914 may store received parameter settings in the local memory subsystem 928 until the parameter settings are modified by new input data received from the programmer module 802. The CPU 906 may use the parameters stored in the local memory to control the pulse generator circuitry 936 to generate a stimulus waveform that is modulated by the high frequency oscillator 918 in a range of 300 MHz to 8 GHz. The resulting RF signal may then be amplified by an RF amplifier 926 and sent through an RF switch 923 to the TX antenna 810 to reach the RX antenna 938 through a depth of tissue.

In some implementations, the RF signal sent by the TX antenna 810 may simply be a power transmission signal used by tissue stimulator 814 to generate electric pulses. In other implementations, the RF signal sent by the TX antenna 810 may be a telemetry signal that provides instructions about various operations of the tissue stimulator 814. The telemetry signal may be sent by the modulation of the carrier signal through the skin. The telemetry signal is used to modulate the carrier signal (e.g., a high frequency signal) that is coupled to the antenna 938 and does not interfere with the input received on the same lead to power the tissue stimulator 814. In some embodiments, the telemetry signal and the powering signal are combined into one signal, where the RF telemetry signal is used to modulate the RF powering signal such that the tissue stimulator 814 is powered directly by the received telemetry signal. Separate subsystems in the tissue stimulator 814 harness the power contained in the signal and interpret the data content of the signal.

The RF switch 923 may be a multipurpose device (e.g., a dual directional coupler) that passes the relatively high amplitude, extremely short duration RF pulse to the TX antenna 810 with minimal insertion loss, while simultaneously providing two low-level outputs to the feedback subsystem 912. One output delivers a forward power signal to the feedback subsystem 912, where the forward power signal is an attenuated version of the RF pulse sent to the TX antenna 810, and the other output delivers a reverse power signal to a different port of the feedback subsystem 912, where reverse power is an attenuated version of the reflected RF energy from the TX Antenna 810.

During the on-cycle time (e.g., while an RF signal is being transmitted to tissue stimulator 814), the RF switch 923 is set to send the forward power signal to feedback subsystem 912. During the off-cycle time (e.g., while an RF signal is not being transmitted to the tissue stimulator 814), the RF switch 923 can change to a receiving mode in which the reflected RF energy and/or RF signals from the tissue stimulator 814 are received to be analyzed in the feedback subsystem 912.

The feedback subsystem 912 of the RF pulse generator module 806 may include reception circuitry to receive and extract telemetry or other feedback signals from tissue stimulator 814 and/or reflected RF energy from the signal sent by TX antenna 810. The feedback subsystem 912 may include an amplifier 926, a filter 924, a demodulator 922, and an A/D converter 920. The feedback subsystem 912 receives the forward power signal and converts this high-frequency AC signal to a DC level that can be sampled and sent to the controller subsystem 914. In this way, the characteristics of the generated RF pulse can be compared to a reference signal within the controller subsystem 914. If a disparity (e.g., an error) exists in any parameter, the controller subsystem 914 can adjust the output to the RF pulse generator 806. The nature of the adjustment can be proportional to the computed error. The controller subsystem 914 can incorporate additional inputs and limits on its adjustment scheme, such as the signal amplitude of the reverse power and any predetermined maximum or minimum values for various pulse parameters.

The reverse power signal can be used to detect fault conditions in the RF-power delivery system. In an ideal condition, when TX antenna 810 has perfectly matched impedance to the tissue that it contacts, the electromagnetic waves generated from the RF pulse generator module 806 pass unimpeded from the TX antenna 810 into the body tissue. However, in real-world applications, a large degree of variability exists in the body types of users, types of clothing worn, and positioning of the antenna 810 relative to the body surface. Since the impedance of the antenna 810 depends on the relative permittivity of the underlying tissue and any intervening materials and on an overall separation distance of the antenna 810 from the skin, there can be an impedance mismatch at the interface of the TX antenna 810 with the body surface in any given application. When such a mismatch occurs, the electromagnetic waves sent from the RF pulse generator module 806 are partially reflected at this interface, and this reflected energy propagates backward through the antenna feed.

The dual directional coupler RF switch 923 may prevent the reflected RF energy propagating back into the amplifier 926, and may attenuate this reflected RF signal and send the attenuated signal as the reverse power signal to the feedback subsystem 912. The feedback subsystem 912 can convert this high-frequency AC signal to a DC level that can be sampled and sent to the controller subsystem 914. The controller subsystem 914 can then calculate the ratio of the amplitude of the reverse power signal to the amplitude of the forward power signal. The ratio of the amplitude of reverse power signal to the amplitude level of forward power may indicate severity of the impedance mismatch.

In order to sense impedance mismatch conditions, the controller subsystem 914 can measure the reflected-power ratio in real time, and according to preset thresholds for this measurement, the controller subsystem 914 can modify the level of RF power generated by the RF pulse generator module 806. For example, for a moderate degree of reflected power the course of action can be for the controller subsystem 914 to increase the amplitude of RF power sent to the TX antenna 810, as would be needed to compensate for slightly non-optimum but acceptable TX antenna coupling to the body. For higher ratios of reflected power, the course of action can be to prevent operation of the RF pulse generator module 806 and set a fault code to indicate that the TX antenna 810 has little or no coupling with the body. This type of reflected power fault condition can also be generated by a poor or broken connection to the TX antenna 810. In either case, it may be desirable to stop RF transmission when the reflected power ratio is above a defined threshold, because internally reflected power can lead to unwanted heating of internal components, and this fault condition means that the system cannot deliver sufficient power to the tissue stimulator 814 and thus cannot deliver therapy to the user.

The controller 942 of the tissue stimulator 814 may transmit informational signals, such as a telemetry signal, through the RX antenna 538 to communicate with the RF pulse generator module 806 during its receive cycle. For example, the telemetry signal from the tissue stimulator 814 may be coupled to the modulated signal on the RX antenna 938, during the on and off state of the transistor circuit to enable or disable a waveform that produces the corresponding RF bursts necessary to transmit to the external (or remotely implanted) pulse generator module 806. The RX antenna 938 may be connected to electrodes 954 in contact with tissue to provide a return path for the transmitted signal. An A/D converter can be used to transfer stored data to a serialized pattern that can be transmitted on the pulse modulated signal from the RX antenna 938 of the tissue stimulator 814.

A telemetry signal from the tissue stimulator 814 may include stimulus parameters, such as the power or the amplitude of the current that is delivered to the tissue from the electrodes 954. The feedback signal can be transmitted to the RF pulse generator module 806 to indicate the strength of the stimulus at the target nerve tissue by means of coupling the signal to the RX antenna 938, which radiates the telemetry signal to the RF pulse generator module 806. The feedback signal can include either or both an analog and digital telemetry pulse modulated carrier signal. Data such as stimulation pulse parameters and measured characteristics of stimulator performance can be stored in an internal memory device within the tissue stimulator 814 and sent on the telemetry signal. The frequency of the carrier signal may be in a range of 300 MHz to 8 GHz.

In the feedback subsystem 912, the telemetry signal can be down modulated using the demodulator 922 and digitized by being processed through the analog to digital (A/D) converter 920. The digital telemetry signal may then be routed to the CPU 930 with embedded code, with the option to reprogram, to translate the signal into a corresponding current measurement in the tissue based on the amplitude of the received signal. The CPU 930 of the controller subsystem 914 can compare the reported stimulus parameters to those held in local memory 928 to verify that the tissue stimulator 814 delivered the specified stimuli to target nerve tissue. For example, if the tissue stimulator 814 reports a lower current than was specified, the power level from the RF pulse generator module 806 can be increased so that the tissue stimulator 814 will have more available power for stimulation. The tissue stimulator 814 can generate telemetry data in real time (e.g., at a rate of 8 kbits per second). All feedback data received from the tissue stimulator 814 can be logged against time and sampled to be stored for retrieval to a remote monitoring system accessible by a health care professional for trending and statistical correlations.

The sequence of remotely programmable RF signals received by the RX antenna 938 may be conditioned into waveforms that are controlled within the tissue stimulator 814 by the control subsystem 942 and routed to the appropriate electrodes 954 that are located in proximity to the target nerve tissue. For instance, the RF signal transmitted from the RF pulse generator module 806 may be received by RX antenna 938 and processed by circuitry, such as waveform conditioning circuitry 940, within the tissue stimulator 814 to be converted into electrical pulses applied to the electrodes 954 through an electrode interface 952. In some implementations, the tissue stimulator 814 includes between two to sixteen electrodes 954.

The waveform conditioning circuitry 940 may include a rectifier 944, which rectifies the signal received by the RX antenna 938. The rectified signal may be fed to the controller 942 for receiving encoded instructions from the RF pulse generator module 806. The rectifier signal may also be fed to a charge balance component 946 that is configured to create one or more electrical pulses such that the one or more electrical pulses result in a substantially zero net charge at the one or more electrodes 954 (that is, the pulses are charge balanced). The charge balanced pulses are passed through the current limiter 948 to the electrode interface 952, which applies the pulses to the electrodes 954 as appropriate.

The current limiter 948 ensures the current level of the pulses applied to the electrodes 954 is not above a threshold current level. In some implementations, an amplitude (for example, a current level, a voltage level, or a power level) of the received RF pulse directly determines the amplitude of the stimulus. In this case, it may be particularly beneficial to include current limiter 948 to prevent excessive current or charge being delivered through the electrodes 954, although the current limiter 548 may be used in other implementations where this is not the case. Generally, for a given electrode 954 having several square millimeters of surface area, it is the charge per phase that should be limited for safety (where the charge delivered by a stimulus phase is the integral of the current). But, in some cases, the limit can instead be placed on the current, where the maximum current multiplied by the maximum possible pulse duration is less than or equal to the maximum safe charge. More generally, the current limiter 948 acts as a charge limiter that limits a characteristic (for example, a current or duration) of the electrical pulses so that the charge per phase remains below a threshold level (typically, a safe-charge limit).

In the event the tissue stimulator 814 receives a “strong” pulse of RF power sufficient to generate a stimulus that would exceed the predetermined safe-charge limit, the current limiter 948 can automatically limit or “clip” the stimulus phase to maintain the total charge of the phase within the safety limit. The current limiter 948 may be a passive current limiting component that cuts the signal to the electrodes 954 once the safe current limit (the threshold current level) is reached. Alternatively, or additionally, the current limiter 948 may communicate with the electrode interface 952 to turn off all electrodes 954 to prevent tissue damaging current levels.

A clipping event may trigger a current limiter feedback control mode. The action of clipping may cause the controller to send a threshold power data signal to the RF pulse generator module 806. The feedback subsystem 912 detects the threshold power signal and demodulates the signal into data that is communicated to the controller subsystem 914. The controller subsystem 914 algorithms may act on this current-limiting condition by specifically reducing the RF power generated by the RF pulse generator module 806, or cutting the power completely. In this way, the RF pulse generator module 806 can reduce the RF power delivered to the body if the tissue stimulator 814 reports that it is receiving excess RF power.

The controller 950 may communicate with the electrode interface 952 to control various aspects of the electrode setup and pulses applied to the electrodes 954. The electrode interface 952 may act as a multiplex and control the polarity and switching of each of the electrodes 954. For instance, in some implementations, the tissue stimulator 814 has multiple electrodes 954 in contact with the target neural tissue, and for a given stimulus, the RF pulse generator module 806 can arbitrarily assign one or more electrodes to act as a stimulating electrode, to act as a return electrode, or to be inactive by communication of assignment sent wirelessly with the parameter instructions, which the controller 950 uses to set electrode interface 952 as appropriate. It may be physiologically advantageous to assign, for example, one or two electrodes 954 as stimulating electrodes and to assign all remaining electrodes 954 as return electrodes.

Also, in some implementations, for a given stimulus pulse, the controller 950 may control the electrode interface 952 to divide the current arbitrarily (or according to instructions from the RF pulse generator module 806) among the designated stimulating electrodes. This control over electrode assignment and current control can be advantageous because in practice the electrodes 954 may be spatially distributed along various neural structures, and through strategic selection of the stimulating electrode location and the proportion of current specified for each location, the aggregate current distribution on the target neural tissue can be modified to selectively activate specific neural targets. This strategy of current steering can improve the therapeutic effect for the patient.

In another implementation, the time course of stimuli may be arbitrarily manipulated. A given stimulus waveform may be initiated at a time T_start and terminated at a time T_final, and this time course may be synchronized across all stimulating and return electrodes. Furthermore, the frequency of repetition of this stimulus cycle may be synchronous for all of the electrodes 954. However, the controller 950, on its own or in response to instructions from the RF pulse generator module 806, can control electrode interface 952 to designate one or more subsets of electrodes to deliver stimulus waveforms with non-synchronous start and stop times, and the frequency of repetition of each stimulus cycle can be arbitrarily and independently specified.

For example, a tissue stimulator 814 having eight electrodes 954 may be configured to have a subset of five electrodes, called set A, and a subset of three electrodes, called set B. Set A may be configured to use two of its electrodes as stimulating electrodes, with the remainder being return electrodes. Set B may be configured to have just one stimulating electrode. The controller 950 could then specify that set A deliver a stimulus phase with 3 mA current for a duration of 200 us, followed by a 400 us charge-balancing phase. This stimulus cycle could be specified to repeat at a rate of 60 cycles per second. Then, for set B, the controller 950 could specify a stimulus phase with 1 mA current for duration of 500 us, followed by a 800 us charge-balancing phase. The repetition rate for the set B stimulus cycle can be set independently of set A (e.g., at 25 cycles per second). Or, if the controller 950 was configured to match the repetition rate for set B to that of set A, for such a case the controller 950 can specify the relative start times of the stimulus cycles to be coincident in time or to be arbitrarily offset from one another by some delay interval.

In some implementations, the controller 950 can arbitrarily shape the stimulus waveform amplitude, and may do so in response to instructions from the RF pulse generator module 806. The stimulus phase may be delivered by a constant-current source or a constant-voltage source, and this type of control may generate characteristic waveforms that are static. For example, a constant current source generates a characteristic rectangular pulse in which the current waveform has a very steep rise, a constant amplitude for the duration of the stimulus, and then a very steep return to baseline. Alternatively, or additionally, the controller 950 can increase or decrease the level of current at any time during the stimulus phase and/or during the charge-balancing phase. Thus, in some implementations, the controller 950 can deliver arbitrarily shaped stimulus waveforms such as a triangular pulse, sinusoidal pulse, or Gaussian pulse for example. Similarly, the charge-balancing phase can be arbitrarily amplitude-shaped, and similarly a leading anodic pulse (prior to the stimulus phase) may also be amplitude-shaped.

As described above, the tissue stimulator 814 may include a charge balancing component 946. Generally, for constant current stimulation pulses, pulses should be charge balanced by having the amount of cathodic current should equal the amount of anodic current, which is typically called biphasic stimulation. Charge density is the amount of current times the duration it is applied, and is typically expressed in the units uC/cm². In order to avoid the irreversible electrochemical reactions such as pH change, electrode dissolution as well as tissue destruction, no net charge should appear at the electrode-electrolyte interface, and it is generally acceptable to have a charge density less than 30 uC/cm². Biphasic stimulating current pulses ensure that no net charge appears at the electrode 954 after each stimulation cycle and that the electrochemical processes are balanced to prevent net dc currents. The tissue stimulator 814 may be designed to ensure that the resulting stimulus waveform has a net zero charge. Charge balanced stimuli are thought to have minimal damaging effects on tissue by reducing or eliminating electrochemical reaction products created at the electrode-tissue interface.

A stimulus pulse may have a negative-voltage or current, called the cathodic phase of the waveform. Stimulating electrodes may have both cathodic and anodic phases at different times during the stimulus cycle. An electrode 954 that delivers a negative current with sufficient amplitude to stimulate adjacent neural tissue is called a “stimulating electrode.” During the stimulus phase, the stimulating electrode acts as a current sink. One or more additional electrodes act as a current source and these electrodes are called “return electrodes.” Return electrodes are placed elsewhere in the tissue at some distance from the stimulating electrodes. When a typical negative stimulus phase is delivered to tissue at the stimulating electrode, the return electrode has a positive stimulus phase. During the subsequent charge-balancing phase, the polarities of each electrode are reversed.

In some implementations, the charge balance component 946 uses one or more blocking capacitors placed electrically in series with the stimulating electrodes and body tissue, between the point of stimulus generation within the stimulator circuitry and the point of stimulus delivery to tissue. In this manner, a resistor-capacitor (RC) network may be formed. In a multi-electrode stimulator, one charge-balance capacitors may be used for each electrode, or a centralized capacitors may be used within the stimulator circuitry prior to the point of electrode selection. The RC network can block direct current (DC). However, the RC network can also prevent low-frequency alternating current (AC) from passing to the tissue. The frequency below which the series RC network essentially blocks signals is commonly referred to as the cutoff frequency, and in some embodiments, the design of the stimulator system may ensure that the cutoff frequency is not above the fundamental frequency of the stimulus waveform. In the example embodiment 800, the tissue stimulator 814 may have a charge-balance capacitor with a value chosen according to the measured series resistance of the electrodes and the tissue environment in which the stimulator is implanted. By selecting a specific capacitance value, the cutoff frequency of the RC network in this embodiment is at or below the fundamental frequency of the stimulus pulse.

In other implementations, the cutoff frequency may be chosen to be at or above the fundamental frequency of the stimulus, and in this scenario the stimulus waveform created prior to the charge-balance capacitor, called the drive waveform, may be designed to be non-stationary, where the envelope of the drive waveform is varied during the duration of the drive pulse. For example, in one embodiment, the initial amplitude of the drive waveform is set at an initial amplitude Vi, and the amplitude is increased during the duration of the pulse until it reaches a final value k*Vi. By changing the amplitude of the drive waveform over time, the shape of the stimulus waveform passed through the charge-balance capacitor is also modified. The shape of the stimulus waveform may be modified in this fashion to create a physiologically advantageous stimulus.

In some implementations, the tissue stimulator 814 may create a drive-waveform envelope that follows the envelope of the RF pulse received by the RX antenna 938. In this case, the RF pulse generator module 806 can directly control the envelope of the drive waveform within the tissue stimulator 814, and thus no energy storage may be required inside of the tissue stimulator 814, itself. In this implementation, the stimulator circuitry may modify the envelope of the drive waveform or may pass it directly to the charge-balance capacitor and/or electrode-selection stage.

In some implementations, the tissue stimulator 814 may deliver a single-phase drive waveform to the charge balance capacitor or it may deliver multiphase drive waveforms. In the case of a single-phase drive waveform (e.g., a negative-going rectangular pulse), this pulse comprises the physiological stimulus phase, and the charge-balance capacitor is polarized (charged) during this phase. After the drive pulse is completed, the charge balancing function is performed solely by the passive discharge of the charge-balance capacitor, where is dissipates its charge through the tissue in an opposite polarity relative to the preceding stimulus. In one implementation, a resistor within the tissue stimulator 814 facilitates the discharge of the charge-balance capacitor. In some implementations, using a passive discharge phase, the capacitor may allow virtually complete discharge prior to the onset of the subsequent stimulus pulse.

In the case of multiphase drive waveforms, the tissue stimulator 814 may perform internal switching to pass negative-going or positive-going pulses (phases) to the charge-balance capacitor. These pulses may be delivered in any sequence and with varying amplitudes and waveform shapes to achieve a desired physiological effect. For example, the stimulus phase may be followed by an actively driven charge-balancing phase, and/or the stimulus phase may be preceded by an opposite phase. Preceding the stimulus with an opposite-polarity phase, for example, can have the advantage of reducing the amplitude of the stimulus phase required to excite tissue.

In some implementations, the amplitude and timing of stimulus and charge-balancing phases is controlled by the amplitude and timing of RF pulses from the RF pulse generator module 806, and in other implementations, this control may be administered internally by circuitry onboard the tissue stimulator 814, such as controller 550. In the case of onboard control, the amplitude and timing may be specified or modified by data commands delivered from the pulse generator module 806.

While the RF pulse generator module 806 and the TX antenna 810 have been described and illustrated as separate components, in some embodiments, the RF pulse generator module 806 and the TX antenna 810 may be physically located in the same housing or other packaging. Furthermore, while the RF pulse generator module 806 and the TX antenna 810 have been described and illustrated as located external to the body, in some embodiments, either or both of the RF pulse generator module 806 and the TX antenna 810 may be designed to be implanted subcutaneously. While the RF pulse generator module 806 and the TX antenna 810 have been described and illustrated as coupled via a wired connection 808, in some embodiments (e.g., where the RF pulse generator module 806 is either located externally or implanted subcutaneously), the RF pulse generator module 806 and the TX antenna 810 may be coupled via a wireless connection.

While the above-discussed implantation devices, tools, and techniques, tissue stimulators 100, 814, and neural stimulation system 800 have been described and illustrated with respect to certain dimensions, sizes, shapes, arrangements, materials, and methods, in some embodiments, an implantation device, tool, or technique, a tissue stimulator, or a neural stimulation system that is otherwise substantially similar in construction and function to any of the above-discussed embodiments may include one or more different dimensions, sizes, shapes, arrangements, and materials or may be utilized according to different methods.

Accordingly, other embodiments are also within the scope of the following claims.

What is claimed is:

1. A method of percutaneously implanting a tissue stimulator at a tissue of a body, the method comprising:
inserting a sheath of an implantation device into the body through an incision adjacent the tissue;
inserting the tissue stimulator into the sheath to position one or more fixation mechanisms of the tissue stimulator near the tissue within the body;
with at least a portion of the sheath inside of the body:
separating the sheath into two or more portions, and
moving the two or more portions apart from each other to remove the sheath from the tissue stimulator; and
withdrawing the two or more portions of the sheath from the body through the incision.

2. A method of percutaneously implanting a tissue stimulator into a body, the method comprising:
inserting a distal end of the tissue stimulator through an incision near a tissue to be stimulated;
positioning electrodes of the tissue stimulator carried at the distal end of the tissue stimulator adjacent the tissue to be stimulated;
inserting a sheath of an implantation device into the body through the incision;
inserting a proximal end of the tissue stimulator into the sheath to position fixation mechanisms of the tissue stimulator in an appropriate tissue within the body;
with at least a portion of the sheath inside of the body, breaking the sheath apart into two or more portions to remove the sheath from the tissue stimulator; and
withdrawing the two or more portions of the sheath from the body through the incision.

3. An implantation system comprising:
a tissue stimulator comprising a fixation module configured to anchor the tissue stimulator to a tissue; and
an implantation device comprising a sheath that defines:
a passage for receiving the tissue stimulator to position the tissue stimulator at the tissue,
a curved interior surface at an opening of the passage for forcing the fixation module of the tissue stimulator into a collapsed configuration in which the tissue stimulator is movable through the passage, and
a handle by which the sheath is breakable into two or more portions for removing the sheath from the tissue stimulator to allow the fixation module to adjust to an extended configuration in which the fixation module can grip the tissue to anchor the tissue stimulator to the tissue.
